(19) United States
(12) Patent Application Publication
(10) Pub. No.: US 2008/0114171 A1
(43) Pub. Date: May 15, 2008

(54) Solifenacin base forms and preparation thereof

(76) Inventors: Tamas Koltai, Netanya (IL); Nurit Perlman, Kfar Saba (IL); Tamar Nidam, Yehud (IL); Mili Abramov, Givataim (IL)

Correspondence Address: KENYON & KENYON LLP, ONE BROADWAY, NEW YORK, NY, 10004, US

(21) Appl. No.: 11/890,316

(22) Filed: Aug. 3, 2007

Related U.S. Application Data
(60) Provisional application No. 60/835,806, filed on Aug. 3, 2006.
(60) Provisional application No. 60/845,260, filed on Sep. 18, 2006.
(60) Provisional application No. 60/845,261, filed on Sep. 18, 2006.
(60) Provisional application No. 60/859,951, filed on Nov. 20, 2006.
(60) Provisional application No. 60/859,952, filed on Nov. 20, 2006.
(60) Provisional application No. 60/878,913, filed on Jan. 4, 2007.
(60) Provisional application No. 60/898,789, filed on Jan. 31, 2007.
(60) Provisional application No. 60/898,888, filed on Jan. 31, 2007.
(60) Provisional application No. 60/930,391, filed on May 15, 2007.
(60) Provisional application No. 60/949,112, filed on Jul. 11, 2007.

(51) Int. Cl. C07D 453/02; A61P 13/10

(52) U.S. Cl. 546/137

(57) ABSTRACT

Polymorphic forms of solifenacin base have been prepared and characterized. These polymorphic forms are particularly useful for preparing solifenacin salts.

### CROSS-REFERENCE TO RELATED APPLICATIONS

This application claims the benefit of Provisional Application Ser. No. 60/835,806, filed Aug. 3, 2006, Provisional Application Ser. No. 60/845,260, filed Sep. 18, 2006, Provisional Application Ser. No. 60/845,261, filed Sep. 18, 2006, Provisional Application Ser. No. 60/859,951, filed Nov. 20, 2006, Provisional Application Ser. No. 60/859,952, filed Nov. 20, 2006, Provisional Application Ser. No. 60/878,913, filed Jan. 4, 2007, Provisional Application Ser. No. 60/898,789, filed Jan. 31, 2007, Provisional Application Ser. No. 60/898,888, filed Jan. 31, 2007, Provisional Application Ser. No. 60/930,391, filed May 15, 2007, and to Provisional Application Ser. No. 60/949,112, filed Jul. 11, 2007. The contents of these applications are incorporated herein in their entirety by reference.

### FIELD OF THE INVENTION

The present invention relates to amorphous and crystalline forms of solifenacin base and to the preparation thereof.

### BACKGROUND OF THE INVENTION

Solifenacin base of the following formula

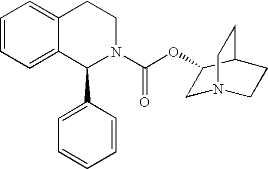

C₂₃H₂₆N₂O₂

Exact Mass: 362.1994

Mol. Wt.: 362.4647

m/e: 362. 1994 (100.0%), 363.2028 (25.6%), 364.2061 (3.1%)

C, 76.21; H, 7.23; N, 7.73; 0, 8.83,

is the key intermediate of solifenacin salts such as solifenacin succinate. Solifenacin succinate, (3R)-1-azabicyclo[2.2.2]oct-3-yl-(1S)-1-phenyl-3,4-dihydroisoquinoline-2-(1H)-carboxylate-succinate, or (S)-Phenyl-1,2,3,4-tetrahydroisoquinoline-2-carboxylic acid 3(R)-quinuclidinyl ester succinate, of the chemical structure

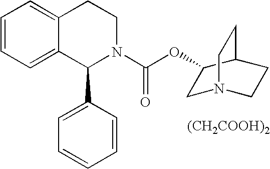

, is a urinary antispasmodic indicated for the treatment of urge incontinence and/or increased urinary frequency and urgency as may occur in patients with overactive bladder syndrome (OAB). The drug is marketed under the name Vesicare® in 5 mg and 10 mg tablets.

Solifenacin and derivatives thereof, as well as salts thereof, are reportedly encompassed in U.S. Pat. No. 6,017,927.

Solifenacin base is described in J. Med. Chem. (2005) 48(21), 6597-6606 as colorless oil. WO 2005/105795 reportedly encompasses a substance containing solifenacin or solifenacin itself.

Polymorphism, the occurrence of different solid state forms, is a property of some molecules and molecular complexes. A single molecule, like solifenacin base, may give rise to a variety of solid states forms having distinct crystal structures and physical properties such as melting point, powder x-ray diffraction (“PXRD”) pattern, infrared (“IR”) absorption fingerprint, and solid state nuclear magnetic resonance (“NMR”) spectrum. One solid state form may give rise to thermal behavior different from that of another solid state form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different solid state forms results from the orientation and intermolecular interactions of adjacent molecules or complexes in the bulk solid. Accordingly, polymorphs are distinct solids sharing the same molecular formula yet having distinct advantageous physical properties compared to other solid state forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different solid state forms or polymorphs of the same pharmaceutical compounds can and reportedly do have different aqueous solubilities.

The discovery of new polymorphic forms of solifenacin base provides a new opportunity to improve the performance of the active pharmaceutical ingredient (“API”), solifenacin succinate, by producing solid state forms of solifenacin base having improved characteristics, such as stability, flowability, and solubility. Thus, there is a need in the art for polymorphic forms of solifenacin base.

### SUMMARY OF THE INVENTION

In one embodiment, the invention encompasses solifenacin base in solid form.

In one embodiment, the invention encompasses an amorphous form of solifenacin base. The amorphous form of solifenacin base may be characterized by a PXRD pattern substantially as depicted in FIG. 1.

Optionally, the above amorphous form of solifenacin base contains not more than about 10 wt %, preferably not more than about 5 wt %, more preferably not more than about 1 wt % of the crystalline form of solifenacin base characterized by PXRD peaks at about 7.7, 9.9, 16.2, and 20.9°±0.2° 2θ. Preferably, the above amorphous form of solifenacin base contains not more than about 10 wt %, preferably not more than about 5 wt %, more preferably not more than about 1 wt % of any single crystalline form of solifenacin base.

In another embodiment, the invention encompasses a process for preparing amorphous solifenacin base comprising reacting a solifenacin salt with an inorganic base.

In one embodiment, the invention encompasses a crystalline form of solifenacin base characterized by PXRD peaks at about 5.5, 13.2, 15.8, and 20.6°±0.2° 2θ.

Optionally, the above crystalline form of solifenacin base contains not more than about 10 wt %, preferably not more than about 5 wt %, more preferably not more than about 1 wt % of the crystalline form of solifenacin base characterized by PXRD peaks at about 7.7, 9.9, 16.2, and 20.9°±0.2° 2θ. Preferably, the above crystalline form of solifenacin base contains not more than about 10 wt %, preferably not more than about 5 wt %, and more preferably not more than about 1 wt % of any other single crystalline form of solifenacin base.

In another embodiment, the invention encompasses a process for preparing a crystalline form of solifenacin base characterized by PXRD peaks at about 5.5, 13.2, 15.8, and 20.6°±0.2° 2θ, comprising slurrying solifenacin base in diisopropylether.

In one embodiment, the invention encompasses a crystalline form of solifenacin base characterized by X-ray powder diffraction peaks at about 7.7, 9.9, 16.2, and 20.9°±0.2° 2θ.

Optionally, the above crystalline form of solifenacin base contains not more than about 10 wt %, preferably not more than about 5 wt % and more preferably not more than about 1 wt % of the crystalline form of solifenacin base characterized by PXRD peaks at about 5.5, 13.2, 15.8, and 20.6°±0.2° 2θ. Preferably, the above crystalline form of solifenacin base contains not more than about 10 wt %, preferably not more than about 5 wt %, and more preferably not more than about 1 wt % of any other single crystalline form of solifenacin base.

In another embodiment, the invention encompasses a process for preparing crystalline form of solifenacin base characterized by X-ray powder diffraction peaks at about 7.7, 9.9, 16.2, and 20.9°±0.2° 2θ, comprising

(a) reacting 1(S)-phenyl-1,2,3,4-tetraisoquinoline ethyl carbamate (“(S)-IQL ethyl carbamate”) with 3(R)-quinuclidinol (“(R)-QNC)” in the presence of a base and a first organic solvent;

(b) adding water to obtain a first two-phase system;

(c) separating the phases of the first two-phase system;

(d) adding acidic water to the organic phase from the first two-phase system to obtain a second two-phase system;

(e) separating the phases of the second two phase system;

(f) adding a second organic solvent and an inorganic base to the aqueous phase from the third two-phase system;

(g) separating the phases of the third two-phase system; and

(h) drying the organic phase separated from the third two phase system to obtain solifenacin base.

In one embodiment, the invention encompasses a process for preparing solifenacin salts, comprising preparing any one of the amorphous form of solifenacin base, the crystalline form of solifenacin base characterized by PXRD peaks at about 5.5, 13.2, 15.8, and 20.6°±0.2° 2θ, and crystalline form of solifenacin base characterized by X-ray powder diffraction peaks at about 7.7, 9.9, 16.2, and 20.9°±0.2° 2θ, and converting it to solifenacin salt.

### BRIEF DESCRIPTION OF THE DRAWINGS

FIG. 1 illustrates a characteristic PXRD pattern of the amorphous form of solifenacin base.

FIG. 2 illustrates a characteristic PXRD pattern of solifenacin base crystalline form characterized by PXRD peaks at about 5.5, 13.2, 15.8, and 20.6°±0.2° 2θ.

FIG. 3 illustrates a characteristic PXRD pattern of solifenacin base crystalline form characterized by PXRD peaks at about 7.7, 9.9, 16.2, and 20.9°±0.2° 2θ.

### DETAILED DESCRIPTION OF THE INVENTION

As used herein, the term “room temperature” or “RT” refers the ambient temperature of a typical laboratory, which is usually about 15° C. to about 30° C., often about 18° C. to about 25° C.

As used herein, the term “reflux temperature” refers to the boiling point of the solvent or mixture being heated.

As used herein, the term “vacuum” or “reduced pressure” refers to a pressure of about to 2 mmHg to about 100 mmHg.

As used herein, the term “PXRD” refers to powder X-ray diffraction, the term “IR” refers to infrared, the term “NMR” refers to nuclear magnetic resonance, the term “TGA” refers to thermogravimetric analysis, and the term “DSC” refers to differential scanning calorimetry.

As used herein, the term “(S)-IQL ethyl carbamate” refers to 1(S)-phenyl-1,2,3,4-tetraisoquinoline ethyl carbamate, the term “(R)-QNC” refers to 3(R)-quinuclidinol, the term “EtOAc” refers to ethyl acetate, the term “DCM” refers to dichloromethane, the term “MTBE” refers to methyltertbutyl ether, and the term “NaOMe” refers to alkoxide.

As used herein, the term “acidic water” refers to water with a pH of less than about 7.

The invention encompasses solifenacin base in solid form.

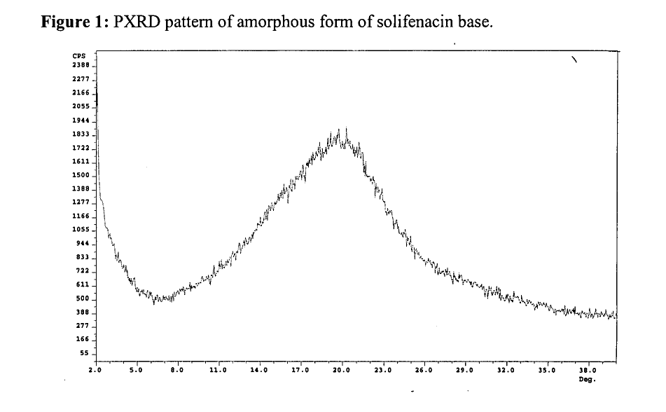

The invention further encompasses an amorphous form of solifenacin base. The amorphous form of solifenacin base may be characterized by a PXRD pattern substantially as depicted in FIG. 1.

Optionally, the above amorphous form of solifenacin base contains not more than about 10 wt %, preferably not more than about 5 wt %, more preferably not more than about 1 wt % of the crystalline form of solifenacin base characterized by PXRD peaks at about 7.7, 9.9, 16.2, and 20.9°±0.2° 2θ. The weight percentage of the crystalline form of solifenacin base characterized by PXRD peaks at about 7.7, 9.9, 16.2, and 20.9°±0.2° 2θ may be calculated based on the percentages of area under the PXRD peaks.

Optionally, the amorphous form of solifenacin base contains not more than about 10 wt %, preferably not more than about 5 wt %, more preferably not more than about 1 wt % of any single crystalline form of solifenacin base. The weight percentages of the crystalline forms of solifenacin base may be calculated based on the percentages of area under the PXRD peaks.

The invention encompasses a process for preparing amorphous solifenacin base comprising reacting a solifenacin salt with an inorganic base.

Preferably, the reaction is performed by dissolving solifenacin salt in water to form a solution, and combining the solution with the inorganic base to form a reaction mixture.

Preferably, the process further comprises adding a water-immiscible organic solvent to obtain a two phase system, extracting the solifenacin base generated into the water-immiscible organic phase, and separating the phases to obtain an organic phase containing a mixture of solifenacin base and a water-immiscible organic solvent.

Preferably, the solifenacin salt is solifenacin succinate.

Optionally, the water immiscible organic solvent is added before or after the inorganic base is combined with the solution of solifenacin salt in water.

Preferably, the water-immiscible organic solvent is selected from the group consisting of halogenated aliphatic hydrocarbon, aromatic hydrocarbon, ester, halogenated aromatic hydrocarbon, and mixtures thereof. Preferably, the ester is selected from the group consisting of ethyl acetate, methyl acetate, butyl acetate, isopropyl acetate, and mixtures thereof. Preferably, the halogenated aromatic hydrocarbon is chlorobenzene. Preferably, the aromatic hydrocarbon is toluene. Preferably, the halogenated aliphatic hydrocarbon is selected from the group consisting of dichloromethane, chloroform, and mixtures thereof. Preferably, the water-immiscible organic solvent is selected from the group consisting of dichloromethane, toluene, and mixtures thereof.

Preferably, the inorganic base is selected from the group consisting of metal hydroxides, metal carbonates, metal bicarbonates, and mixtures thereof. Preferably, the metal hydroxide is selected from the group consisting of lithium hydroxide, sodium hydroxide, potassium hydroxide, and cesium hydroxide. More preferably, the metal hydroxide is NaOH. Preferably, the metal carbonate is selected from sodium carbonate and potassium carbonate. More preferably, the metal carbonate is sodium carbonate. Preferably, the metal bicarbonate is selected from sodium bicarbonate and potassium bicarbonate. Preferably, the inorganic base is NaOH.

The inorganic base may be provided as a solid or in an aqueous solution. Preferably, the inorganic base is provided in an aqueous solution.

Preferably, combining the inorganic base with the solution of solifenacin in water provides a reaction mixture having a pH of about 7 to about 14, more preferably of about 11 to about 14.

Optionally, the process further comprises recovering amorphous solifenacin base from the organic phase. Optionally, the organic phase may be washed with water. Optionally, the organic phase is in a slurry form. The amorphous solifenacin base may be recovered from the slurry by any method known in the art, for example, filtering the slurry to recover the water-immiscible organic phase and removing the solvent.

The recovering step may include removing the water-immiscible organic solvent. Preferably, the removal is by evaporation, more preferably under reduced pressure.

Optionally, after removing the water-immiscible organic solvent, an additional step of slurrying the solifenacin base in ether may be performed. Preferably, the ether is selected from the group consisting of diisopropylether, methyltertbutyl ether, diethylether, and mixtures thereof. More preferably, the ether is diisopropylether. Optionally, the slurry is maintained for sufficient time to obtain amorphous solifenacin base. Preferably, the slurry is maintained for about 4 to about 24 hours, more preferably for about 6 to about 10 hours. Preferably, the slurry is maintained at a temperature of about 0° C. to about 30° C., more preferably at about 20° C. to about 25° C.

Preferably, the obtained amorphous solifenacin base is in solid form.

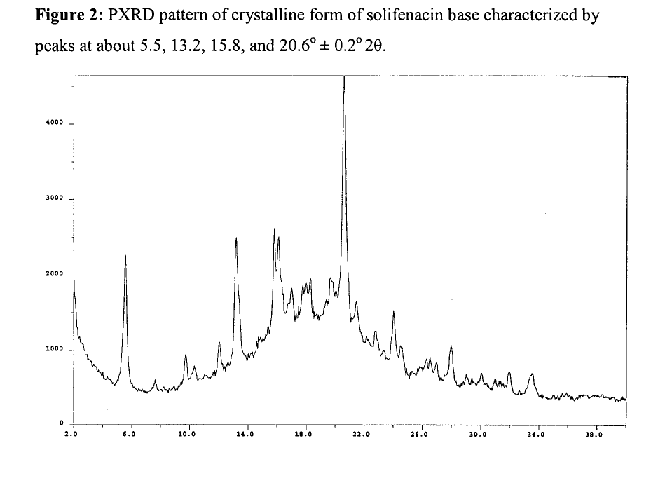

The invention encompasses a crystalline form of solifenacin base (denominated “Form B1”) characterized by PXRD peaks at about 5.5, 13.2, 15.8, and 20.6°±0.2° 2θ. The crystalline form may be further characterized by PXRD peaks at about 9.7, 12.0, 16.1, 17.0, 19.7 and 24.0°±0.2° 2θ. The crystalline form may be further characterized by the PXRD pattern substantially as depicted in FIG. 2.

Optionally, the above crystalline form of solifenacin base characterized by PXRD peaks at about 5.5, 13.2, 15.8, and 20.6°±0.2° 2θ contains not more than about 10 wt %, preferably not more than about 5 wt %, and more preferably not more than about 1 wt % of the crystalline form of solifenacin base characterized by PXRD peaks at about 7.7, 9.9, 16.2, and 20.9°±0.2° 2θ. The weight percentages of the crystalline forms may be calculated based on the area percentages of the PXRD peaks, for example peaks at 15.3 and 20.9°±0.2° 2θ.

Optionally, the above crystalline form of solifenacin base characterized by PXRD peaks at about 5.5, 13.2, 15.8, and 20.6°±0.2° 2θ contains not more than about 10 wt %, preferably not more than about 5 wt %, and more preferably not more than about 1 wt % of any other single crystalline form of solifenacin base.

The invention encompasses a process for preparing a crystalline form of solifenacin base characterized by PXRD peaks at about 5.5, 13.2, 15.8, and 20.6°±0.2° 2θ, comprising slurrying solifenacin base in diisopropylether.

Optionally, the starting solifenacin base is amorphous solifenacin base prepared according to the process described above. Optionally, the starting solifenacin base is prepared from reaction between (S)-IQL ethyl carbamate and (R)-QNC.

Preferably, prior to the slurrying step, the solifenacin base is extracted from an organic solvent selected from EtOAc and DCM.

Optionally, the process further comprises recovering the crystalline form of solifenacin base. Optionally, the recovery step comprises isolating the crystalline form by filtering and drying it. Preferably, the drying is for about 10 hours to about 24 hours. Preferably, the drying is performed at a temperature of about 40° C. to about 60° C. Preferably, the drying is performed under vacuum.

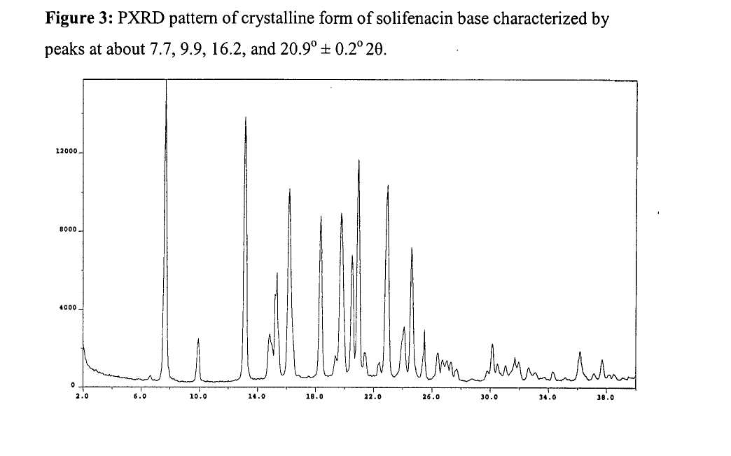

The invention encompasses a crystalline form of solifenacin base (denominated “Form B2”) characterized by PXRD peaks at about 7.7, 9.9, 16.2, and 20.9°±0.2° 2θ. The crystalline form may be further characterized by PXRD peaks at about 15.3, 18.3, 19.8, and 22.9°±0.2° 2θ. The crystalline form may be further characterized by the PXRD pattern substantially as depicted in FIG. 3.

Optionally, the above crystalline form of solifenacin base characterized by PXRD peaks at about 7.7, 9.9, 16.2, and 20.9°±0.2° 2θ contains not more than about 10 wt %, preferably not more than about 5 wt %, and more preferably not more than about 1 wt % of the crystalline form of solifenacin base characterized by PXRD peaks at about 5.5, 13.2, 15.8, and 20.6°±0.2° 2θ. The weight percentages of the crystalline forms may be calculated based on the area percentages of the PXRD peaks, for example peaks at 5.5 and 15.8°±0.2° 2θ.

Optionally, the above crystalline form of solifenacin base characterized by PXRD peaks at about 7.7, 9.9, 16.2, and 20.9°±0.2° 2θ contains not more than about 10 wt %, preferably not more than about 5 wt %, and more preferably not more than about 1 wt % of any other single crystalline form of solifenacin base.

The invention encompasses a process for preparing crystalline form of solifenacin base characterized by PXRD peaks at about 7.7, 9.9, 16.2, and 20.9°±0.2° 2θ, comprising:

(a) reacting (S)-IQL ethyl carbamate with (R)-QNC in the presence of a base and a first organic solvent;

(b) adding water to obtain a first two-phase system;

(c) separating the phases of the first two-phase system;

(d) adding acidic water to the organic phase from the first two-phase system to obtain a second two-phase system;

(e) separating the phases of the second two phase system;

(f) adding a second organic solvent and an inorganic base to the aqueous phase from the third two-phase system;

(g) separating the phases of the third two-phase system;

(h) drying the organic phase separated from the third two phase system to obtain solifenacin base.

Optionally, the process further comprises maintaining the solifenacin base obtained from the organic phase separated from the second two phase system for a sufficient period of time at a temperature to obtain the crystalline form of solifenacin base. Preferably, the maintenance is for a period of about 2 hours to about 3 days, more preferably about 5 hours to about 48 hours. Preferably, the maintenance is at room temperature.

Preferably, the molar ratio between the (R)-QNC and the (S)-IQL ethyl carbamate in step (a) is from about 1.2 to about 1.7, more preferably from about 1.2 to about 1.5.

Preferably, the first organic solvent in step (a) is selected from the group consisting of toluene, xylene, and mixture thereof. More preferably, the organic solvent is toluene. Preferably, the ratio between the first organic solvent and the (S) —IQL ethyl carbamate is from about 0.5 to about 3 ml/g, more preferably from about 1 to about 2 ml/g.

Preferably, the base in step (a) is selected from the group consisting of NaH, NaNH₂, metal alkoxide, and mixtures thereof. More preferably, the base is NaH. Preferably, the molar ratio between the base and the (S)-IQL ethyl carbamate is from about 0.15 to about 0.5, more preferably from about 0.15 to about 0.3.

Preferably, the acidic water in step (d) is added to obtain a pH of about 1 to about 4. Preferably, the acid is HCl.

Preferably, the second organic solvent in step (f) is selected from the group consisting of EtOAc, DCM, toluene, and mixtures thereof. More preferably, the organic solvent is EtOAc.

Preferably, the inorganic base in step (f) is selected from the group consisting of NaHCO₃, KHCO₃, K₂CO₃, Na₂CO₃, NaOH, KOH, and mixtures thereof. More preferably, the inorganic base is K₂CO₃.

Optionally, the drying is done by evaporation.

The invention encompasses a process for preparing solifenacin salts, comprising preparing any one of the amorphous form of solifenacin base, the crystalline form of solifenacin base characterized by PXRD peaks at about 5.5, 13.2, 15.8, and 20.6°±0.2° 2θ, and crystalline form of solifenacin base characterized by X-ray powder diffraction peaks at about 7.7, 9.9, 16.2, and 20.9°±0.2° 2θ, and converting it to solifenacin salt.

Preferably, the solifenacin salt is selected from the group consisting of solifenacin oxalate, solifenacin succinate, solifenacin acetate, and solifenacin-HX, wherein X is a halogen atom, preferably Cl. More preferably, the solifenacin salt is solifenacin succinate.

The amorphous form of solifenacin base, the crystalline form of solifenacin base characterized by PXRD peaks at about 5.5, 13.2, 15.8, and 20.6°±0.2° 2θ, and crystalline form of solifenacin base characterized by X-ray powder diffraction peaks at about 7.7, 9.9, 16.2, and 20.9°±0.2° 2θ may be converted to solifenacin salt by reacting the base with an acid, as described, for example, in U.S. patent application Ser. No. 11/645,021, WO 2005/075474, WO 2005/087231, WO 2005/105795, and in J. Med. Chem., 48(21), 2005, pp. 6597-6606, which are incorporated herein by reference. Preferably, the acid is selected from the group consisting of oxalic acid, succinic acid, acetic acid, and HX, wherein X is a halogen atom, preferably Cl. The conversion to solifenacin succinate may be performed by dissolving solifenacin base in organic solvent such as ethanol, ethyl acetate, methylethylketone, isopropylether, isobutylacetate, methylacetate, and MTBE; adding succinic acid; and cooling.

### EXAMPLES

XRD diffraction was performed on Scintag X-ray powder diffractometer model X′TRA with a solid state detector. Copper radiation of 1.5418 Å was used. The sample holder was a round standard aluminum sample holder with rough zero background. The scanning parameters were: range: 2-40° 2θ; scan mode: continuous scan; step size: 0.05 deg.; rate: 5 deg/min.

### Example 1

### Preparation of Amorphous Solifenacin Base

Solifenacin-succinate (40 g) was dissolved in water (100 ml). NaOH solution (47%, 15 ml) was added, the pH was adjusted to 14, and then DCM (200 ml) was added. The phases were separated. The aqueous phase was extracted twice with DCM. The combined organic phase was divided into 10 parts, and each part was evaporated (30 mbar) to dryness at 40° C. to obtain amorphous solifenacin base solid.

### Example 2

### Preparation of Amorphous Solifenacin Base

SLF-succinate (10.4 g) was dissolved in water (25 ml) and toluene (50 ml). NaOH solution (1M, 20 ml and 47%, 2 ml) was added, and the pH was adjusted to 14. The phases were separated. The organic phase was extracted with water and evaporated to dryness to obtain solifenacin base (8.23 g).

Diisopropylether (100 ml) was added, and a sticky turbid slurry appeared. After stirring at RT overnight, the product was isolated by vacuum filtration under N₂ atmosphere to obtain amorphous SLF base solid.

### Example 3

### Preparation of Solifenacin Succinate

Amorphous SLF base (7.2 g) is dissolved in ethanol (28 ml) at room temperature to form a solution. Succinic acid (2.4 g) is then added to the solution to form a mixture. After two hours, the mixture is cooled to 5° C. The resulting precipitate is isolated by vacuum filtration, washed with ethanol (10 ml), and dried in a vacuum oven at 50° C. for 24 hours to obtain solifenacin succinate.

### Example 4

### Preparation of Solifenacin Base Form B1

An EtOAc solution of solifenacin base (prepared according to WO 2005/105795) was evaporated to obtain solifenacin base (40 g) as oil. Diisopropylether (200 ml) was added to the oil residue and stirred at RT overnight. The white solid was isolated by vacuum filtration under N₂ flow, and dried by vacuum oven at 55° C. for 24 hours to obtain solid of solifenacin base crystalline Form B1 (1.5 g).

### Example 5

### Preparation of Solifenacin Base Form B2

A 100 ml round bottom flask equipped with mechanical stirrer, thermometer and Dean-stark condenser was loaded with (S)-IQL-ethyl carbamate (18 g), toluene (45 ml), (R)-QNC (4.07 g), and NaH (60%, 0.77 g). The mixture was heated to reflux and stirred. At t=1, 2, and 3 hours, the mixture was monitored by HPLC for the formation of solifenacin base, and (R)-QNC (4.07 g) was added. After another hour (total 4 hours), the solution was diluted with toluene (10 ml/g of carbamate), and extracted with water (90 ml). The organic phase was extracted with HCl solution (4%, 108 ml). EtOAc (90 ml) and K₂CO₃ (17.64 g) were added to the aqueous layer and the phases were separated.

The product was isolated by drying the EtOAc solution on MgSO₄ and evaporating the solvent to obtain solifenacin base (18.8 g). After a sufficient amount of time the residue has solidified to obtain solifenacin base crystalline Form B2.

### Example 6

### Preparation of Form I of Solifenacin Succinate

Solifenacin base (3.22 g) was dissolved in methylethylketone (30 ml) at room temperature. Then succinic acid (1.1 g) was added. The solution was stirred at room temperature for 18 hrs, during which it became a slurry. The product was isolated by vacuum filtration, washed with methylethylketone (2×5 ml), and dried in a vacuum oven at 50° C. overnight to obtain solifenacin succinate crystalline Form I (1.33 g, 31% yield).

### Example 7

### Preparation of Form I of Solifenacin Succinate

Solifenacin base (2.68 g) was dissolved in isopropylether (30 ml) at room temperature. Then succinic acid (1 g) was added. The solution was stirred at room temperature for 19 hrs, during which it became a slurry. The product was isolated by vacuum filtration, washed with IPA (2×3 ml), and dried in a vacuum oven at 50° C. overnight to obtain solifenacin succinate crystalline Form I (1.5 g, 42% yield).

### Example 8

### Preparation of Form I of Solifenacin Succinate

Solifenacin base (3.3 g) was dissolved in isobutylacetate (30 ml) at room temperature. Then succinic acid (1.1 g) was added. During the addition the solution became a slurry, and it was stirred at room temperature for 3 hrs. The product was isolated by vacuum filtration and dried in a vacuum oven at 50° C. overnight to obtain solifenacin succinate crystalline Form I (1.02 g, 23% yield).

### Example 9

### Preparation of Form II of Solifenacin Succinate

Solifenacin base (3.2 g) was dissolved in methylacetate (30 ml) at room temperature. Then succinic acid (1.1 g) was added, and the solution became a slurry. After 3.5 hrs, the product was isolated by vacuum filtration, washed with methylacetate (2×5 ml), and dried in a vacuum oven at 50° C. overnight to obtain solifenacin succinate crystalline Form II (2.94 g, 69% yield).

### Example 10

### Preparation of Form II of Solifenacin Succinate

Solifenacin base (3.26 g) was dissolved in MTBE (45 ml) at room temperature. Then succinic acid (1.1 g) was added, and the solution became slurry. After 4 hrs, the product was isolated by vacuum filtration, washed with MTBE (2×5 ml), and dried in a vacuum oven at 50° C. overnight to obtain solifenacin succinate crystalline Form II (3.31 g, 76.6% yield).

### Example 11

### Preparation of Solifenacin Base

A 100 ml round bottom flask equipped with mechanical stirrer, thermometer, and Dean-stark condenser was loaded with (S)-IQL-ethyl carbamate (25 g), xylene (25 ml), (R)-QNC (16.93 g), and NaH (60%, 0.53 g). The mixture was heated to reflux and stirred. The mixture was monitored by HPLC every hour. After 3 hours, the solution was diluted with xylene (225 ml), and extracted with water (125 ml). The organic phase was extracted with HCl solution (4%, 150 ml). EtOAc (150 ml) and K₂CO₃ (24.5 g) were added to the aqueous layer, and the phases were separated. The solution was dried on MgSO₄ and evaporated to obtain solifenacin base (29 g).

### Example 12

### Preparation of Solifenacin Base Form B2

A 100 ml round bottom flask equipped with mechanical stirrer, thermometer, and Dean-stark condenser was loaded with (S)-IQL-ethyl carbamate (25 g), toluene (25 ml), (R)-QNC (16.96 g), and NaNH₂ (1.04 g). The mixture was heated to reflux and stirred. The mixture was monitored by HPLC for the formation of solifenacin base. After 8 hours the solution was diluted with toluene (9 ml/g of carbamate), and extracted with water (5 ml/g of carbamate). The organic phase was extracted with HCl solution (4%, 6 ml/g of carbamate). EtOAc (6 ml/g of carbamate) and K₂CO₃ (24.5 g) were added to the aqueous layer, and the phases were separated. The solution was dried on MgSO₄ and evaporated to obtain solifenacin base (26.8 g). After a sufficient amount of time the residue has solidified to obtain solifenacin base crystalline Form B2.

1. Solifenacin base in solid form.

2. Amorphous form of solifenacin base.

3. The amorphous form of solifenacin base of claim 2, characterized by a PXRD pattern substantially as depicted in FIG. 1.

4. The amorphous form of solifenacin base of claim 2, containing not more than about 10 wt % of crystalline form of solifenacin base characterized by PXRD peaks at about 7.7, 9.9, 16.2, and 20.9°±0.2° 2θ.

5. The amorphous form of solifenacin base of claim 4, containing not more than about 10 wt % of any other single crystalline form of solifenacin base.

6. The amorphous form of solifenacin base of claim 4, containing not more than about 5 wt % of crystalline form of solifenacin base characterized by PXRD peaks at about 7.7, 9.9, 16.2, and 20.9°±0.2° 2θ.

7. The amorphous form of solifenacin base of claim 6, containing not more than about 5 wt % of any other single crystalline form of solifenacin base.

8. The amorphous form of solifenacin base of claim 6, containing not more than about 1 wt % of crystalline form of solifenacin base characterized by PXRD peaks at about 7.7, 9.9, 16.2, and 20.9°±0.2° 2θ.

9. The amorphous form of solifenacin base of claim 6, containing not more than about 1 wt % of any other single crystalline form of solifenacin base.

10. A process for preparing amorphous solifenacin base of claim 2, comprising reacting a solifenacin salt with an inorganic base.

11. The process of claim 10, wherein the solifenacin salt is solifenacin succinate.

12. The process of claim 10, wherein the inorganic base is selected from the group consisting of metal hydroxides, metal carbonates, metal bicarbonates, and mixtures thereof.

13. The process of claim 12, wherein the metal hydroxide is selected from the group consisting of lithium hydroxide, sodium hydroxide, potassium hydroxide, and cesium hydroxide, the metal carbonate is selected from sodium carbonate and potassium carbonate, and the metal bicarbonate is selected from sodium bicarbonate and potassium bicarbonate.

14. The process of claim 13, wherein the metal hydroxide is sodium hydroxide and the metal carbonate is sodium carbonate.

15. The process of claim 14, wherein the inorganic base is sodium hydroxide.

16. The process of claim 10, wherein the inorganic base is provided in an aqueous solution.

17. The process of claim 10, comprising dissolving solifenacin salt in water to form a solution, and combining the solution with the inorganic base to form a reaction mixture.

18. The process of claim 17, wherein the reaction mixture has a pH of about 7 to about 14.

19. The process of claim 18, wherein the reaction mixture has a pH of about 111 to about 14.

20. The process of claim 17, further comprising adding a water-immiscible organic solvent to obtain a two phase system, extracting the solifenacin base generated into the water-immiscible organic phase, and separating the phases to obtain an organic phase containing a mixture of solifenacin base and a water-immiscible organic solvent.

21. The process of claim 20, wherein the water-immiscible organic solvent is selected from the group consisting of halogenated aliphatic hydrocarbon, aromatic hydrocarbon, ester, halogenated aromatic hydrocarbon, and mixtures thereof.

22. The process of claim 21, wherein the ester is selected from the group consisting of ethyl acetate, methyl acetate, butyl acetate, isopropyl acetate, and mixtures thereof, the halogenated aromatic hydrocarbon is chlorobenzene, the aromatic hydrocarbon is toluene, the halogenated aliphatic hydrocarbon is selected from the group consisting of dichloromethane, chloroform, and mixtures thereof.

23. The process of claim 22, wherein the water-immiscible organic solvent is selected from the group consisting of dichloromethane, toluene, and mixtures thereof.

24. The process of claim 20, wherein the water immiscible organic solvent is added before or after the inorganic base is combined with the solution of solifenacin salt in water.

25. The process of claim 20, further comprising recovering amorphous solifenacin base.

26. The process of claim 25, wherein the water immiscible organic solvent is removed to recover amorphous solifenacin base.

27. The process of claim 26, wherein the water immiscible organic solvent is removed by evaporation.

28. The process of claim 20, further comprising slurrying the solifenacin base in ether.

29. The process of claim 28, wherein the ether is selected from the group consisting of diisopropylether, methyltertbutyl ether, diethylether, and mixtures thereof.

30. The process of claim 29, wherein the ether is diisopropylether.

31. The process of claim 28, wherein the slurry is maintained for about 4 to about 24 hours.

32. The process of claim 31, wherein the slurry is maintained for about 6 to about 10 hours.

33. The process of claim 28, wherein the slurry is maintained at a temperature of about 0° C. to about 30° C.

34. The process of claim 33, wherein the slurry is maintained at about 20° C. to about 25° C.

35. Crystalline form of solifenacin base characterized by PXRD peaks at about 5.5, 13.2, 15.8, and 20.6°±0.2° 2θ.

36-45. (canceled)

46. Crystalline form of solifenacin base characterized by X-ray powder diffraction peaks at about 7.7, 9.9, 16.2, and 20.9°±0.2° 2θ.

47-71. (canceled)

72. A process for preparing solifenacin succinate, comprising converting the solifenacin base of claim 2 to solifenacin succinate.

73. (canceled)
